(19) United States
(12) Patent Application Publication
(10) Pub. No.: US 2010/0063160 A1
(43) Pub. Date: Mar. 11, 2010

(54) POLYMORPHS OF O-DESMETHYL VENLAFAXINE SUCCINATE

(75) Inventors: Vinayak G. Gore, Mararashtra (IN); Vikas S. Kulkarni, Mararashtra (IN); V.S. Wakchaure, Mararashtra (IN); M.G. Hublikar, Mararashtra (IN); S.R. Wavhal, Mararashtra (IN)

Correspondence Address: FULBRIGHT & JAWORSKI L.L.P.;Attn: MN IP Docket, 600 Congress Avenue, Suite 2400, Austin, TX, 78701, US

(73) Assignee: Merck Development Centre Private Limited, Maharashtra (IN)

(21) Appl. No.: 12/445,544

(22) PCT Filed: Oct. 18, 2007
(86) PCT No.: PCT/GB2007/050644

§ 371 Date: Jul. 7, 2009

(30) Foreign Application Priority Data
Oct. 18, 2006 (IN) 1730/MUM/2006

(51) Int. Cl. A61K 31/137; C07C 215/42; A61P 25/24; A61P 25/22

(52) U.S. Cl. 514/649; 564/360

(57) ABSTRACT

The present invention relates to crystalline forms of O-desmethyl venlafaxine (ODV) succinate monohydrate, namely Forms I and II, in pure form and to novel processes for their preparation. The present invention provides a process for the preparation of Form II free of Form I and a process for the preparation of Form I free of Form II. The present invention provides direct methods for the preparation of ODV succinate Form II from ODV free base and for the preparation of ODV succinate Form I from ODV free base.

### CROSS-REFERENCE TO RELATED APPLICATIONS

This application is a Section 371 National Stage Application of International No. PCT/GB2007/050644, filed 18 Oct. 2007 and published as WO 2008/047167 A1 on 24 Apr. 2008, which claims priority from the India Application 1730/mum/2006, filed 18 Oct. 2006, the contents of which are incorporated herein in their entirety for all purposes.

### FIELD OF THE INVENTION

The present invention relates to crystalline forms of O-desmethyl venlafaxine (ODV) succinate monohydrate, namely Forms I and II, in pure form and to novel processes for their preparation. The present invention provides a process for the preparation of Form II free of Form I and a process for the preparation of Form I free of Form II. The present invention provides direct methods for the preparation of ODV succinate Form II from ODV free base and for the preparation of ODV succinate Form I from ODV free base.

### BACKGROUND OF THE INVENTION

Venlafaxine is known to be an antidepressant. O-Desmethyl venlafaxine (ODV), chemically named 1-[2-(dimethylamino)-1-(4-hydroxyphenyl)ethyl]cyclohexanol, is a major metabolite of venlafaxine. ODV has been shown to inhibit norepinephrine and serotonin uptake. Various patents describe processes for the preparation of ODV free base, which can be converted into desired salts, such as ODV succinate.

Preparing a salt or a polymorph of a known compound is a means of altering the physiochemical and biological characteristics of that compound. This is advantageous in pharmaceutical dosage form development.

Polymorphism influences every aspect of the solid state properties of a drug and one of the important aspects of polymorphism in pharmaceuticals is the possibility of interconversion among polymorphic forms. Polymorphic impurity can cause variation of properties relevant to the use, efficacy, stability etc. of pharmaceutically important substances.

For pharmaceuticals in which the active ingredient can exist in more than one polymorphic form, it is particularly important to ensure that the manufacturing process for the active ingredient affords a single polymorph with a consistent level of polymorphic purity. Inconsistencies in polymorphic form and/or polymorphic purity of an active ingredient can lead to inconsistencies in dissolution and/or bioavailability in the pharmaceutical compositions which can lead to batches of active ingredient or pharmaceutical compositions having to be discarded.

ODV succinate, shown below, is well absorbed in the gastrointestinal tract and provides optimal properties for pharmaceutical formulation due to its high solubility, permeability and bioavailability.

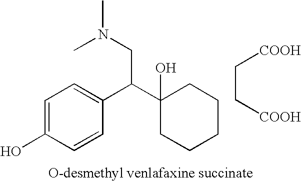

U.S. Pat. No. 6,673,838 reports that oral administration of ODV succinate, in particular in sustained release formulations, results in a lower incidence of nausea, vomiting, diarrhea, abdominal pain, headache, vasovagal malaise and/or trismus than oral administration of venlafaxine. Consequently, ODV succinate is effective in treating patients suffering from depression, anxiety, panic disorder etc. The treatment method includes administering to a patient in need thereof an effective amount of ODV succinate or a substantially pure polymorph of ODV succinate.

U.S. Pat. No. 6,673,838 describes five polymorphs of ODV succinate (four crystalline polymorphs and one amorphous polymorph) and processes for their preparation. There are two crystalline monohydrate forms (Form I and II), one crystalline hydrate form (Form III with a water content between hemi- and monohydrate), one crystalline anhydrate form (Form IV) and one amorphous form.

U.S. Pat. No. 6,673,838 describes a process for the preparation of Form I from ODV base. It also reports the preparation of Form II from Form I as well as from ODV base. The processes reported for the preparation of Form I and Form II from ODV base use the same solvents, namely acetone and water. The two processes, i.e. the process for Form I and the process for Form II, only differ in the volume of solvents used. The patent mentions that during the preparation of Form I, the reaction mixture becomes clear to cloudy, and that during the preparation of Form II, the reaction mixture becomes a clear solution. It is difficult to understand these observations, because in the experiment where a higher volume of solvents was used the solution was clear to cloudy. Moreover, from almost identical experimental conditions two different polymorphic forms were apparently isolated. The parameter, controlling which particular polymorphic form (Form I or Form II) is produced, is not clear and hence impossible to reproduce. Therefore these processes suffer from the serious drawback of lack of selectivity and the small variation in process parameters can lead to the isolation of an undesired form or an inconsistent mixture of two forms or more.

Therefore, there remains a need for an improved process for the consistent and reproducible formation of ODV succinate Form II with a very high chemical and polymorphic purity.

### SUMMARY OF THE INVENTION

The present invention provides direct methods for the preparation of ODV

Form I and Form II from ODV free base. The present invention has the advantage of providing the ODV succinate salt Form I and Form II with little or no polymorphic impurity of other forms, as they are prepared directly from ODV base. The processes of the present invention do not involve any interconversion of polymorphic forms unlike the processes disclosed in U.S. Pat. No. 6,673,838 Therefore the possibility of obtaining polymorphic impurities is minimized or even eliminated. Moreover the present invention reports different solvent systems for the preparation processes of Form I and Form II and, consequently, this further reduces or eliminates the amount of any polymorphic impurity.

The present invention provides processes for the preparation of essentially pure ODV succinate Form I and Form II, which can be easily adopted for commercial production with a high degree of consistency with respect to polymorphic composition.

The present inventors have found that Form II of ODV succinate is the most stable form.

Accordingly a first aspect of the present invention provides ODV succinate Form II substantially free of other polymorphs. The first aspect of the present invention also provides ODV succinate Form I substantially free of other polymorphs.

A polymorphic form is ‘substantially free’ of other polymorphic forms, if it contains less than 10% by weight of other polymorphic forms, preferably less than 5% by weight, preferably less than 2% by weight, preferably less than 1% by weight, preferably less than 0.5% by weight.

A second aspect of the present invention provides a direct method for the preparation of ODV succinate Form II from ODV free base. The second aspect of the present invention also provides a direct method for the preparation of ODV succinate Form I from ODV free base.

A ‘direct method’ for the preparation of ODV succinate Form II from ODV free base converts ODV free base to ODV succinate Form II without proceeding via any other polymorphic ODV succinate forms. Likewise, a ‘direct method’ for the preparation of ODV succinate Form I from ODV free base converts ODV free base to ODV succinate Form I without proceeding via any other polymorphic ODV succinate forms.

A third aspect of the present invention provides a method for the preparation of ODV succinate Form II without involving any interconversion of ODV succinate polymorphic forms. The third aspect of the present invention also provides a method for the preparation of ODV succinate Form I without involving any interconversion of ODV succinate polymorphic forms.

A fourth aspect of the present invention provides a process for the preparation of ODV succinate Form II from ODV free base, comprising the steps of:

(a) reacting O-desmethyl venlafaxine and succinic acid in (i) toluene and water, (ii) methanol, (iii) methanol and acetone, (iv) water, or (v) acetonitrile and N,N-dimethylformamide;

(b) cooling the reaction mixture;

(c) filtering the reaction mixture to obtain a solid product; and

(d) drying the solid product.

Preferably step (a)(i) is carried out at a temperature in the range of 55° C. to 115° C., preferably 70° C. to 115° C., preferably 100° C. to 115° C. Preferably step (a)(i) is carried until a clear solution is obtained.

Preferably step (a)(ii) is carried out at a temperature in the range of 55° C. to 65° C., preferably 60° C. to 65° C. Preferably step (a)(ii) is carried out for about 30 minutes, preferably until a clear solution is obtained.

Preferably step (a)(iii) is carried out at a temperature in the range of 55° C. to 65° C., preferably 58° C. to 62° C. Preferably step (a)(iii) is carried out for about 30 minutes, preferably until a clear solution is obtained.

Preferably step (a)(iv) is carried out at a temperature in the range of 55° C. to 100° C., preferably 80° C. to 100° C., preferably 95° C. to 100° C. Preferably step (a)(iv) is carried out for about 30 minutes, preferably until a clear solution is obtained.

Preferably step (a)(v) is carried out at a temperature in the range of 55° C. to 115° C., preferably 70° C. to 100° C. Preferably step (a)(v) is carried out for about 1 hour, preferably until a clear solution is obtained.

Preferably the reaction mixture is cooled in step (b) to 5° C. to 30° C., preferably to 10° C. to 30° C., preferably to 20° C. to 30° C., preferably to 25° C. to 30° C., preferably to about 26° C.

If methanol, or methanol and acetone, are used in step (a), then preferably after the cooling of step (b), the following steps are carried out: (b1) the solvent(s) is/are removed by vacuum distillation, (b2) dichloromethane is added and then removed by vacuum distillation, and (b3) further dichloromethane is added to produce a slurry. Step (b2) may be carried out once, twice, three times or more.

In step (d), the solid product is preferably dried at 50° C. to 80° C., preferably at 50° C. to 70° C. Preferably the solid product is dried under vacuum, preferably a vacuum of about 0.8 kg/cm². Preferably the solid product is dried until a constant weight is obtained.

A fifth aspect of the present invention provides a process for the preparation of ODV succinate Form I from ODV free base, comprising the steps of:

(a) reacting O-desmethyl venlafaxine and succinic acid in N,N-dimethyl-formamide, acetone and water;

(b) cooling the reaction mixture;

(c) filtering the reaction mixture to obtain a solid product; and

(d) drying the solid product.

Preferably step (a) is carried out at a temperature in the range of 60° C. to 90° C., preferably 70° C. to 90° C., preferably 80° C. to 90° C. Preferably step (a) is carried out for 0.5 to 5 hours, preferably for about 1 hour.

Preferably the reaction mixture is cooled in step (b) to 5° C. to 30° C., preferably 10° C. to 30° C., preferably 20° C. to 30° C., preferably to about 26° C.

In step (d), the solid product is preferably dried at 50° C. to 80° C., preferably at 60° C. to 80° C., preferably at about 70° C. Preferably the solid product is dried under vacuum, preferably a vacuum of about 0.8 kg/cm². Preferably the solid product is dried until a constant weight is obtained.

The method or process of the present invention is preferably carried out on a commercial scale, preferably to obtain batches of ODV succinate Form I or Form II in the order of 0.5 kg, 1 kg, 5 kg, 10 kg, 50 kg or more.

A sixth aspect of the present invention provides ODV succinate Form II when prepared by a process according to the present invention. The sixth aspect of the present invention also provides ODV succinate Form I when prepared by a process according to the present invention.

A seventh aspect of the present invention provides ODV succinate according to the present invention for use in medicine. Preferably the ODV succinate is for treating or preventing depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence, or Parkinson's disease.

An eighth aspect of the present invention provides a pharmaceutical composition comprising ODV succinate according to the present invention.

A ninth aspect of the present invention provides a use of ODV succinate according to the present invention, for the manufacture of a medicament for the treatment or prevention of depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence, or Parkinson's disease.

A tenth aspect of the present invention provides a method of treating or preventing depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence, or Parkinson's disease, comprising administering a therapeutically or prophylactically effective amount of ODV succinate according to the present invention to a patient in need thereof. Preferably the patient is a mammal, preferably a human. Preferably the amount of the ODV succinate administered is from 0.01 mg to 50 mg per kg per day.

### DETAILED DESCRIPTION OF THE INVENTION

The present invention provides novel methods for the preparation of ODV succinate Form I and Form II directly from ODV free base without interconversion of polymorphic forms.

Succinic acid salts of ODV exist as enantiomers and the present invention includes racemic mixtures as well as stereoisomerically pure forms of the same. The term ‘ODV succinate’ as used herein refers to racemic mixtures and stereoisomerically pure forms of ODV succinate, unless otherwise indicated. The term ‘stereoisomerically pure’ refers to compounds, which are comprised of a greater proportion of the desired isomer than of the optical antipode. A stereoisomerically pure compound is generally made up of at least 80%, preferably at least 90%, preferably at least 95%, more preferably at least 99%, of the desired isomer based upon 100% total weight of ODV succinate salt.

The present invention relates to ODV succinate Form I and Form II, which are crystalline monohydrate salts. The processes disclosed in this application are capable of providing ODV succinate Form I and Form II in consistent chemical and polymorphic purity irrespective of the scale of preparation.

A further aspect of the present invention is pure ODV succinate Form I free of any other polymorphic form and pure ODV succinate Form II free of any other polymorphic form. The term ‘pure’ when used herein means that the pure polymorphic form contains less than 10% by weight of another polymorphic form. Preferably, the pure polymorphic form contains less than 5% by weight of another polymorphic form. More preferably, the pure polymorphic form contains less than 2% by weight of another polymorphic form. Most preferably, the pure polymorphic form contains less than 1% by weight of another polymorphic form.

According to another aspect of the present invention there is provided a novel direct method for the preparation of ODV succinate Form II from ODV free base. The said process provides ODV succinate Form II in substantially pure form free of other polymorphs. A further aspect of the present invention is a direct method for the preparation of ODV succinate Form I and Form II from

ODV base without any interconversion of polymorphic forms. Another aspect of the present invention is to provide an alternate process for the preparation of ODV succinate Form I.

Further aspects of the present invention are pharmaceutical compositions comprising the polymorphs and uses of the pharmaceutical compositions in methods of treating patients suffering from depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence, or Parkinson's disease, the treatment methods comprising providing to a patient an effective amount of ODV succinate. Additionally, ODV succinate can be administered to treat hypothalamic amenorrhea in depressed and non-depressed human females.

Further aspects of the present invention are compositions of the pure ODV Form I and Form II along with pharmaceutically acceptable excipient(s). Other aspects of the present invention are the pharmaceutical compositions containing ODV Form I and Form II and uses of the pharmaceutical compositions in methods of treating patients suffering from depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence, or Parkinson's disease.

The details of the invention, its objects and advantages are explained hereunder in greater detail in relation to non-limiting exemplary illustrations.

### EXAMPLES

### Example 1

O-Desmethyl venlafaxine (25 g) and succinic acid (11.25 g) were added to a mixture of N,N-dimethylformamide (50 ml), acetone (125 ml) and water (1.72 ml).

The reaction mixture was heated to 83-85° C. and stirred at 83-85° C. 1 hour before cooling to 26° C. Filtration of the reaction mixture afforded the product as an off-white solid. The solid product was dried at 70° C. under 0.8 kg/cm² vacuum until a constant weight was obtained (weight of the product=23.5 g).

The ¹H-NMR indicated formation of ODV succinate. The TGA indicated that the ODV succinate salt formed was a hydrate. The XRPD and DSC analysis data confirmed that the product obtained was the Form I of ODV succinate hydrate.

### Example 2

O-Desmethyl venlafaxine (2 g) and succinic acid (0.92 g) were added to toluene (40 ml). The reaction mixture was heated to reflux at 112° C. To the clear toluene solution, water (10 ml) was added at 100° C. The resulting biphasic mixture was cooled to 25-30° C. with stirring. The succinate salt precipitated and was isolated as an off-white solid by filtration. The solid product was dried at 50° C. under 0.8 kg/cm² vacuum until a constant weight was obtained (weight of the product=1.8 g).

The ¹H-NMR indicated formation of ODV succinate. The TGA indicated that the ODV succinate salt formed was a hydrate. The XRPD and DSC analysis data confirmed that the product obtained was the Form II of ODV succinate hydrate.

### Example 3

O-Desmethyl venlafaxine (10 g) followed by succinic acid (4.6 g) were added to methanol (250 ml) and the reaction mixture was heated to reflux (65° C.) and stirred for 30 minutes at 65° C. The clear solution was allowed to cool to 26° C. and the methanol was removed by vacuum distillation. Dichloromethane (150 ml) was added to the residual oil and was then distilled off. This process was repeated again with dichloromethane (150 ml), whereupon the sticky mass changed into a free flowing off-white solid. The product was isolated as an off-white solid by filtration from a slurry in dichloromethane. The solid product was dried at 50° C. under 0.8 kg/cm² vacuum until a constant weight was obtained (weight of the product=8.5 g).

The ¹H-NMR indicated formation of ODV succinate. The TGA indicated that the ODV succinate salt formed was a hydrate. The XRPD and DSC analysis data confirmed that the product obtained was the Form II of ODV succinate hydrate.

### Example 4

O-Desmethyl venlafaxine (10 g) followed by succinic acid (4.6 g) were added to a mixture of methanol (50 ml) and acetone (150 ml). The white suspension was heated to reflux (60° C.) and the reaction mixture was stirred for 30 minutes at 60° C. The resultant clear solution was allowed to cool to 26° C. and the solvent was then removed by vacuum distillation. Dichloromethane (150 ml) was added to the residual oil and was then distilled off. This process was repeated again with dichloromethane (150 ml), whereupon the sticky mass changed into a free flowing off-white solid. The product was isolated as an off-white solid by filtration from a slurry in dichloromethane. The solid product was dried at 50° C. under 0.8 kg/cm² vacuum until a constant weight was obtained (weight of the product=8.0 g).

The ¹H-NMR indicated formation of ODV succinate. The TGA indicated that the ODV succinate salt formed was a hydrate. The XRPD and DSC analysis data confirmed that the product obtained was the Form II of ODV succinate hydrate.

### Example 5

O-Desmethyl venlafaxine (2 g) followed by succinic acid (0.92 g) were added to water (16 ml) and the suspension was heated to reflux (100° C.) and stirred for 30 minutes at 100° C. The clear solution was allowed to cool to 26° C. and the reaction mixture was filtered to obtain the product as an off-white solid. The solid product was dried at 50° C. under 0.8 kg/cm² vacuum until a constant weight was obtained (weight of the product=1.7 g).

The ¹H-NMR indicated formation of ODV succinate. The TGA indicated that the ODV succinate salt formed was a hydrate. The XRPD and DSC analysis data confirmed that the product obtained was the Form II of ODV succinate hydrate.

### Example 6

O-Desmethyl venlafaxine (2 g) followed by succinic acid (0.92 g) were added to a mixture of acetonitrile (16 ml) and N,N-dimethylformamide (4 ml) and the suspension was heated to 70° C. The clear solution was heated at 100° C. for 1 hour before cooling to 26° C. The reaction mixture was then filtered to obtain the product as an off-white solid. The solid product was dried at 70° C. under 0.8 kg/cm² vacuum until a constant weight was obtained (weight of the product=1.65 g).

The ¹H-NMR indicated formation of ODV succinate. The TGA indicated that the ODV succinate salt formed was a hydrate. The XRPD and DSC analysis data confirmed that the product obtained was the Form II of ODV succinate hydrate.

It will be understood that the present invention has been described above by way of example only. The examples are not intended to limit the scope of the invention. Various modifications and embodiments can be made without departing from the scope and spirit of the invention, which is defined by the following claims only.

What is claimed is:

1. O-desmethyl venlafaxine succinate Form I or Form II substantially free of other polymorphs.

2. A direct method for the preparation of O-desmethyl venlafaxine (ODV) succinate Form I or Form II from ODV free base.

3. A method for the preparation of O-desmethyl venlafaxine (ODV) succinate Form I or Form II without involving any interconversion of ODV succinate polymorphic forms.

4. A process for the preparation of O-desmethyl venlafaxine (ODV) succinate Form II from ODV free base, comprising the steps of:
(a) reacting O-desmethyl venlafaxine and succinic acid in (i) toluene and water, (ii) methanol, (iii) methanol and acetone, (iv) water, or (v) acetonitrile and N,N-dimethylformamide;
(b) cooling the reaction mixture;
(c) filtering the reaction mixture to obtain a solid product; and
(d) drying the solid product.

5. The process as claimed in claim 4, wherein:
a. step (a) is carried out in toluene and water at a temperature in the range of 55° C. to 115° C.; or
b. step (a) is carried out in methanol at a temperature in the range of 55° C. to 65° C.; or
c. step (a) is carried out in methanol and acetone at a temperature in the range of 55° C. to 65° C.; or
d. step (a) is carried out in water at a temperature in the range of 55° C. to 100° C.; or
e. step (a) is carried out in acetonitrile and N,N-dimethylformamide at a temperature in the range of 55° C. to 115° C.; and/or
f. in step (b) the reaction mixture is cooled to 20° C. to 30° C.; and/or
g. the ODV succinate Form II is prepared on a commercial scale.

6. A process for the preparation of O-desmethyl venlafaxine (ODV) succinate Form I from ODV free base, comprising the steps of:
(a) reacting O-desmethyl venlafaxine and succinic acid in N,N-dimethyl-formamide, acetone and water;
(b) cooling the reaction mixture;
(c) filtering the reaction mixture to obtain a solid product; and
(d) drying the solid product.

7. The process as claimed in claim 6, wherein:
a. step (a) is carried out at a temperature in the range of 60° C. to 90° C.; and/or
b. in step (b) the reaction mixture is cooled to 20° C. to 30° C.; and/or
c. the ODV succinate Form I is prepared on a commercial scale.

8. O-desmethyl venlafaxine (ODV) succinate Form I or Form II when prepared by a process as claimed in claim 2.

9. O-desmethyl venlafaxine (ODV) succinate as claimed in claim 1, for treating or preventing depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence, or Parkinson's disease.

10. O-desmethyl venlafaxine (ODV) succinate as claimed in claim 8, for treating or preventing depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence, or Parkinson's disease.

11. A pharmaceutical composition comprising ODV succinate as claimed in claim 1 and a pharmaceutically acceptable excipient.

12. A pharmaceutical composition comprising ODV succinate as claimed in claim 8 and a pharmaceutically acceptable excipient.

13. A method of treating or preventing depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence, or Parkinson's disease, comprising administering a therapeutically or prophylactically effective amount of ODV succinate as claimed in claim 1 to a patient in need thereof.

14. The method as claimed in claim 13, wherein the patient is a mammal.

15. The method as claimed in claim 14, wherein the patient is a human.

16. The method as claimed in claim 13, wherein the amount of the ODV succinate administered is from 0.01 mg to 50 mg per kg per day.

17. A method of treating or preventing depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence, or Parkinson's disease, comprising administering a therapeutically or prophylactically effective amount of ODV succinate as claimed in claim 8 to a patient in need thereof.

18. The method as claimed in claim 17, wherein the patient is a mammal.

19. The method as claimed in claim 18, wherein the patient is a human.

20. The method as claimed in claim 17, wherein the amount of the ODV succinate administered is from 0.01 mg to 50 mg per kg per day.
